(19) United States
(12) Patent Application Publication
(10) Pub. No.: US 2025/0057604 A1
(43) Pub. Date: Feb. 20, 2025

(54) Ultrasound Systems and Methods for Sustained Spatial Attention

(71) Applicant: Bard Access Systems, Inc., Salt Lake City, UT (US)

(72) Inventors: Daniel B. Blanchard, Bountiful, UT (US); Zachary S. Davis, Sandy, UT (US)

(21) Appl. No.: 18/936,364

(22) Filed: Nov. 4, 2024

Related U.S. Application Data
(63) Continuation of application No. 17/491,308, filed on Sep. 30, 2021, now Pat. No. 12,137,987.
(60) Provisional application No. 63/086,971, filed on Oct. 2, 2020.

(51) Int. Cl. A61B 34/20; A61B 8/00; A61B 8/08; A61B 17/34

(52) U.S. Cl. CPC A61B 34/20; A61B 8/0841; A61B 8/4254; A61B 8/4488; A61B 8/463; A61B 17/3403; A61B 2017/3405; A61B 2034/2057; A61B 2034/2065

(57) ABSTRACT

An ultrasound probe for sustained spatial attention. The ultrasound probe can include a display screen on a visible side of the ultrasound probe, the display screen coupled to ultrasound imaging components in the ultrasound probe, the ultrasound imaging components configured to capture live subcutaneous images and render the live subcutaneous images on the display screen. The ultrasound probe can further include one or more needle trajectories depicted on the display over the live subcutaneous images, the one or more needle trajectories configured to assist a user in guided insertion of a needle into an anatomical target under the ultrasound probe. The ultrasound probe can further include a light-pattern projector on the visible side of the ultrasound probe adjacent the display screen, the light-pattern projector configured to project a light pattern corresponding to a subcutaneous depth accessible by a needle.

### PRIORITY

This application is a continuation of U.S. patent application Ser. No. 17/491,308, filed Sep. 30, 2021, now U.S. Pat. No. 12,137,987, which claims the benefit of priority to U.S. Provisional Application No. 63/086,971, filed Oct. 2, 2020, each of which is incorporated by reference in its entirety into this application.

### BACKGROUND

A variety of ultrasound systems exist including wired or wireless ultrasound probes for ultrasound imaging. Whether wired or wireless, an ultrasound system such as the foregoing requires a clinician to switch his or her spatial attention between different spatial regions, particularly between 1) a relatively close ultrasound probe being used for ultrasound imaging and 2) a relatively distant display rendering corresponding ultrasound images. Having to switch spatial attention between the ultrasound probe and the display can be difficult when ultrasound imaging and attempting to simultaneously establish an insertion site with a needle, place a vascular access device (“VAD”) such as a catheter in a blood vessel of a patient at the insertion site, or the like. Such difficulties can be pronounced for less experienced clinicians, older clinicians having reduced lens flexibility in their eyes, etc. Ultrasound systems are needed that do not require clinicians to continuously switch their spatial attention between different spatial regions.

Disclosed herein are ultrasound systems and methods for sustained spatial attention in one or more spatial regions.

### SUMMARY

Disclosed herein is an ultrasound probe including, in some embodiments, a probe body, a probe head extending from a distal end of the probe body, and a camera integrated into a side of the ultrasound probe. The probe head includes a plurality of ultrasonic transducers arranged in an array. The camera is configured for recording one or more still or moving images of a procedural field with a depth of field including a plane of a distal end of the probe head and a field of view including a spatial region about the probe head.

In some embodiments, the ultrasound probe further includes a light-pattern projector integrated into the side of the ultrasound probe including the camera. The light-pattern projector is configured to project a light pattern in the spatial region about the probe head focused in the plane of the distal end of the probe head. The light pattern is configured for guided insertion of a needle into an anatomical target under the probe head in the procedural field.

In some embodiments, the light pattern includes periodic hash marks along one or more rays radiating from a central axis of the ultrasound probe in the plane of the probe head. Each hash mark of the hash marks corresponds to a depth under the probe head accessible by the needle along an associated ray at a needle-insertion angle with respect to the plane of the probe head.

In some embodiments, the light pattern includes periodic concentric circular arcs bound between two or more rays radiating from a central axis of the ultrasound probe in the plane of the probe head. Each circular arc of the circular arcs corresponds to a depth under the probe head accessible by the needle along an associated ray at a needle-insertion angle with respect to the plane of the probe head.

In some embodiments, the ultrasound probe further includes a needle-guide holder extending from a side of the probe head in common with the side of the ultrasound probe including the camera.

In some embodiments, the ultrasound probe further includes a single-use needle guide coupled to the needle-guide holder. The needle-guide holder, the needle guide, or a combination of the needle-guide holder and the needle guide includes at least one degree of freedom enabling the needle guide to swivel between sides of the ultrasound probe.

Also disclosed herein is an ultrasound system including, in some embodiments, a console and an ultrasound probe. The console includes a display configured to render on a display screen thereof ultrasound images and one or more still or moving images of a procedural field. The ultrasound probe includes a probe body, a probe head extending from a distal end of the probe body, and a camera integrated into a side of the ultrasound probe. The probe head includes a plurality of ultrasonic transducers arranged in an array. The camera is configured for recording the one-or-more still or moving images of the procedural field with a depth of field including a plane of a distal end of the probe head and a field of view including a spatial region about the probe head.

In some embodiments, the ultrasound probe further includes a needle-guide holder extending from a side of the probe head in common with the side of the ultrasound probe including the camera.

In some embodiments, the ultrasound probe further includes a single-use needle guide coupled to the needle-guide holder. The needle-guide holder, the needle guide, or a combination of the needle-guide holder and the needle guide includes at least one degree of freedom enabling the needle guide to swivel between sides of the ultrasound probe.

In some embodiments, the ultrasound probe further includes a light-pattern projector integrated into the side of the ultrasound probe including the camera. The light-pattern projector is configured to project a light pattern in the spatial region about the probe head focused in the plane of the distal end of the probe head. The light pattern is configured for guided insertion of a needle into an anatomical target under the probe head in the procedural field.

In some embodiments, the light pattern includes periodic hash marks along one or more rays radiating from a central axis of the ultrasound probe in the plane of the probe head. Each hash mark of the hash marks corresponds to a depth under the probe head accessible by the needle along an associated ray at a needle-insertion angle with respect to the plane of the probe head.

In some embodiments, the light pattern includes periodic concentric circular arcs bound between two or more rays radiating from a central axis of the ultrasound probe in the plane of the probe head. Each circular arc of the circular arcs corresponds to a depth under the probe head accessible by the needle along an associated ray at a needle-insertion angle with respect to the plane of the probe head.

In some embodiments, the one-or-more still or moving images show both the light pattern in the spatial region about the probe head and the needle in relation to the light pattern when both the light pattern and the needle are present in the spatial region about the probe head. The one-or-more still or moving images show both the light pattern and the needle in relation to the light pattern for the guided insertion of the needle into the anatomical target under the probe head optionally on the display.

In some embodiments, the display is further configured to render on the display screen one or more overlying needle trajectories lying over the ultrasound images in accordance with one or more depths accessible by the needle indicated by the light pattern. The one-or-more overlying needle trajectories are configured for the guided insertion of the needle into the anatomical target under the probe head on the display.

In some embodiments, the display is further configured to render on the display screen an overlying pattern lying over the one-or-more still or moving images. The overlying pattern is configured for guided insertion of a needle into an anatomical target under the probe head on the display.

In some embodiments, the overlying pattern includes periodic hash marks along one or more rays radiating from a central axis of the ultrasound probe in the plane of the probe head. Each hash mark of the hash marks corresponds to a depth under the probe head accessible by the needle along an associated ray at a needle-insertion angle with respect to the plane of the probe head.

In some embodiments, the overlying pattern includes periodic concentric circular arcs bound between two or more rays radiating from a central axis of the ultrasound probe in the plane of the probe head. Each circular arc of the circular arcs corresponds to a depth under the probe head accessible by the needle along an associated ray at a needle-insertion angle with respect to the plane of the probe head.

In some embodiments, the one-or-more still or moving images show the needle in relation to the overlying pattern when the needle is present in the spatial region about the probe head. The one-or-more still or moving images show the needle in relation to the overlying pattern for the guided insertion of the needle into the anatomical target under the probe head optionally on the display.

In some embodiments, the display is further configured to render on the display screen one or more overlying needle trajectories lying over the ultrasound images in accordance with one or more depths accessible by the needle indicated by the overlying pattern. The one-or-more overlying needle trajectories are configured for the guided insertion of the needle into an anatomical target under the probe head on the display.

Also disclosed herein is an ultrasound probe including, in some embodiments, a probe body, a probe head extending from a distal end of the probe body, and a display integrated into a side of the ultrasound probe. The probe head includes a plurality of ultrasonic transducers arranged in an array. The display is configured to render on a display screen thereof ultrasound images and one or more overlying needle trajectories lying over the ultrasound images. The one-or-more overlying needle trajectories are configured for guided insertion of a needle into an anatomical target under the probe head on the display.

In some embodiments, the ultrasound probe further includes a light-pattern projector integrated into the side of the ultrasound probe including the display. The light-pattern projector is configured to project a light pattern in a spatial region about the probe head focused in a plane of a distal end of the probe head. The light pattern is configured for the guided insertion of the needle into the anatomical target under the probe head in the procedural field.

In some embodiments, the light pattern includes periodic hash marks along one or more rays radiating from a central axis of the ultrasound probe in the plane of the probe head. Each hash mark of the hash marks corresponds to a depth under the probe head accessible by the needle along an associated ray at a needle-insertion angle with respect to the plane of the probe head.

In some embodiments, the light pattern includes periodic concentric circular arcs bound between two or more rays radiating from a central axis of the ultrasound probe in the plane of the probe head. Each circular arc of the circular arcs corresponds to a depth under the probe head accessible by the needle along an associated ray at a needle-insertion angle with respect to the plane of the probe head.

In some embodiments, the one-or-more overlying needle trajectories lying over the ultrasound images are in accordance with one or more depths accessible by the needle indicated by the light pattern.

In some embodiments, the ultrasound probe further includes a needle-guide holder extending from the side of the ultrasound probe including the display.

In some embodiments, the ultrasound probe further includes a single-use needle guide coupled to the needle-guide holder. The needle-guide holder, the needle guide, or a combination of the needle-guide holder and the needle guide includes at least one degree of freedom enabling the needle guide to swivel between sides of the ultrasound probe.

Also disclosed herein is a method of an ultrasound system including, in some embodiments, an ultrasound probe-obtaining step, an ultrasound probe-moving step, a recording step, an ultrasound image-monitoring step, and a needle-inserting step. The ultrasound probe-obtaining step includes obtaining an ultrasound probe. The ultrasound probe includes a probe body, a probe head extending from a distal end of the probe body, and a camera integrated into a side of the ultrasound probe. The ultrasound probe-moving step includes moving the ultrasound probe over a patient while the ultrasound probe emits generated ultrasound signals into the patient from ultrasonic transducers in the probe head and receives reflected ultrasound signals from the patient by the ultrasonic transducers. The recording step includes recording one or more still or moving images of a procedural field with a depth of field including a plane of a distal end of the probe head and a field of view including a spatial region about the probe head. The ultrasound image-monitoring step includes monitoring ultrasound images rendered on a display screen of a display associated with a console of the ultrasound system to identify an anatomical target of the patient under the probe head. The needle-inserting step includes inserting a needle into the anatomical target. Optionally, the inserting of the needle is guided by the display with reference to the one-or-more still or moving images rendered on the display screen thereof.

In some embodiments, the method further includes a needle guide-attaching step. The needle guide-attaching step includes attaching a needle guide to a needle-guide holder extending from the probe body. The needle guide includes a needle through hole configured to direct the needle into the patient under the probe head at a needle-insertion angle with respect to the plane of the probe head.

In some embodiments, the method further includes a needle guide-swiveling step. The needle guide-swiveling step includes swiveling the needle guide between sides of the ultrasound probe to find a suitable needle trajectory before the needle-inserting step. The needle-guide holder, the needle guide, or a combination of the needle-guide holder and the needle guide includes at least one degree of freedom enabling the swiveling of the needle guide.

In some embodiments, the needle is guided in the procedural field during the needle-inserting step in accordance with a light pattern in the spatial region about the probe head. The light pattern is projected from a light-pattern projector integrated into the side of the ultrasound probe including the camera and focused in the plane of the distal end of the probe head for guiding the needle in the procedural field.

In some embodiments, the light pattern includes periodic hash marks along one or more rays radiating from a central axis of the ultrasound probe in the plane of the probe head. Each hash mark of the hash marks corresponds to a depth under the probe head accessible by the needle along an associated ray at a needle-insertion angle with respect to the plane of the probe head.

In some embodiments, the light pattern includes periodic concentric circular arcs bound between two or more rays radiating from a central axis of the ultrasound probe in the plane of the probe head. Each circular arc of the circular arcs corresponds to a depth under the probe head accessible by the needle along an associated ray at a needle-insertion angle with respect to the plane of the probe head.

In some embodiments, the needle is further guided on the display during the needle-inserting step. The one-or-more still or moving images show both the light pattern in the spatial region about the probe head and the needle in relation to the light pattern for guiding the needle on the display.

In some embodiments, the needle is further guided on the display during the needle-inserting step. The ultrasound images show one or more overlying needle trajectories in accordance with one or more depths accessible by the needle indicated by the light pattern for guiding the needle on the display.

In some embodiments, the needle is guided on the display during the needle-inserting step in accordance with an overlying pattern rendered over the one-or-more still or moving images on the display screen for guiding the needle on the display.

In some embodiments, the overlying pattern includes periodic hash marks along one or more rays radiating from a central axis of the ultrasound probe in the plane of the probe head. Each hash mark of the hash marks corresponds to a depth under the probe head accessible by the needle along an associated ray at a needle-insertion angle with respect to the plane of the probe head.

In some embodiments, the overlying pattern includes periodic concentric circular arcs bound between two or more rays radiating from a central axis of the ultrasound probe in the plane of the probe head. Each circular arc of the circular arcs corresponds to a depth under the probe head accessible by the needle along an associated ray at a needle-insertion angle with respect to the plane of the probe head.

In some embodiments, the needle is further guided on the display during the needle-inserting step. The ultrasound images show one or more overlying needle trajectories in accordance with one or more depths accessible by the needle indicated by the overlying pattern for guiding the needle on the display.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates an ultrasound system with a first ultrasound probe in accordance with some embodiments.

FIG. 2 illustrates a perspective view of the ultrasound probe of FIG. 1 in accordance with some embodiments.

FIG. 3 illustrates the ultrasound system with a second ultrasound probe in accordance with some embodiments.

FIG. 4 illustrates a front view of the ultrasound probe of FIG. 3 in accordance with some embodiments.

FIG. 5 illustrates a side view of the ultrasound probe of FIG. 3 in accordance with some embodiments.

FIG. 6 illustrates a schematic of guided insertion of a needle into an anatomical target with a light pattern projected in a procedural field in accordance with some embodiments.

FIG. 7 illustrates a schematic of a first light pattern or first overlying pattern in accordance with some embodiments.

FIG. 8 illustrates a schematic of a second light pattern or a second overlying pattern in accordance with some embodiments.

FIG. 9 illustrates a schematic of a third light pattern or a third overlying pattern in accordance with some embodiments.

FIG. 10 illustrates guided insertion of a needle into an anatomical target with the first light pattern or the first overlying pattern over one or more still or moving images on a display in accordance with some embodiments.

FIG. 11 illustrates guided insertion of a needle into an anatomical target with the third light pattern or the third overlying pattern over the one-or-more still or moving images on the display in accordance with some embodiments.

FIG. 12 illustrates a block diagram of the ultrasound system in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or “proximal section” of, for example, a catheter includes a portion or section of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal section, or the proximal length of the catheter can include the proximal end of the catheter; however, the proximal portion, the proximal section, or the proximal length of the catheter need not include the proximal end of the catheter. That is, unless context suggests otherwise, the proximal portion, the proximal section, or the proximal length of the catheter is not a terminal portion or terminal length of the catheter.

With respect to “distal,” a “distal portion” or a “distal section” of, for example, a catheter includes a portion or section of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal section, or the distal length of the catheter can include the distal end of the catheter; however, the distal portion, the distal section, or the distal length of the catheter need not include the distal end of the catheter. That is, unless context suggests otherwise, the distal portion, the distal section, or the distal length of the catheter is not a terminal portion or terminal length of the catheter.

Unless defined otherwise, all technical and scientific terms used herein have the same meaning as commonly understood by those of ordinary skill in the art.

As set forth above, a variety of ultrasound systems exist including wired or wireless ultrasound probes for ultrasound imaging. Whether wired or wireless, an ultrasound system such as the foregoing requires a clinician to switch his or her spatial attention between different spatial regions, particularly between 1) a relatively close ultrasound probe being used for ultrasound imaging and 2) a relatively distant display rendering corresponding ultrasound images. Having to switch spatial attention between the ultrasound probe and the display can be difficult when ultrasound imaging and attempting to simultaneously establish an insertion site with a needle, place a VAD such as a catheter in a blood vessel of a patient at the insertion site, or the like. Such difficulties can be pronounced for less experienced clinicians, older clinicians having reduced lens flexibility in their eyes, etc. Ultrasound systems are needed that do not require clinicians to continuously switch their spatial attention between different spatial regions.

Disclosed herein are ultrasound systems and methods for sustained spatial attention. For example, an ultrasound system can include a console and an ultrasound probe. A display of the console can be configured to display ultrasound images and one or more still or moving images of a procedural field. The ultrasound probe can include a camera integrated into the ultrasound probe for recording the one-or-more still or moving images of the procedural field with a depth of field including a distal end of a probe head and a field of view including a spatial region about the probe head. With the one-or-more still or moving images displayed along with the ultrasound images, a clinician need not switch his or her spatial attention between spatial regions such as the procedural field and the display quite as frequently as with existing ultrasound systems, thereby sustaining spatial attention in one or more spatial regions. These and other features will become more apparent to those of skill in the art in view of the accompanying drawings and following description, which describe particular embodiments in greater detail.

### Ultrasound Systems

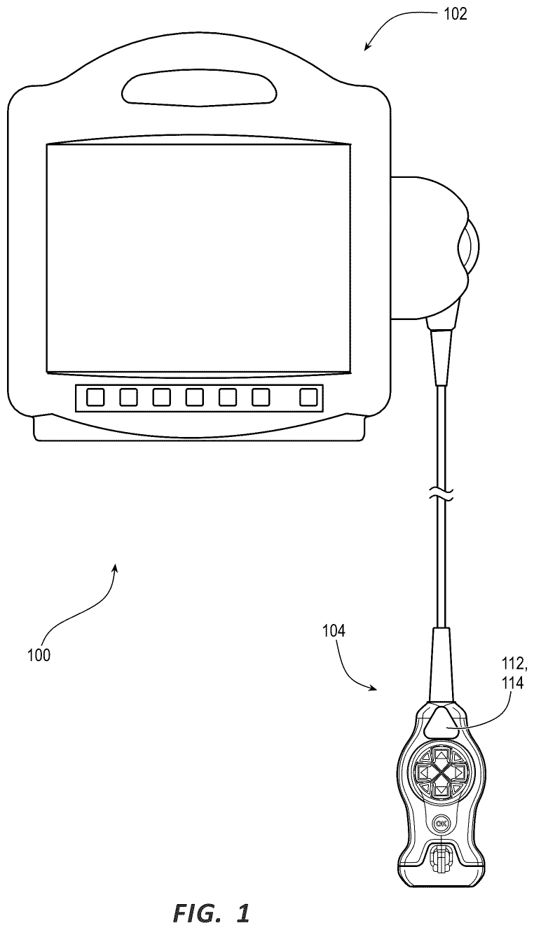

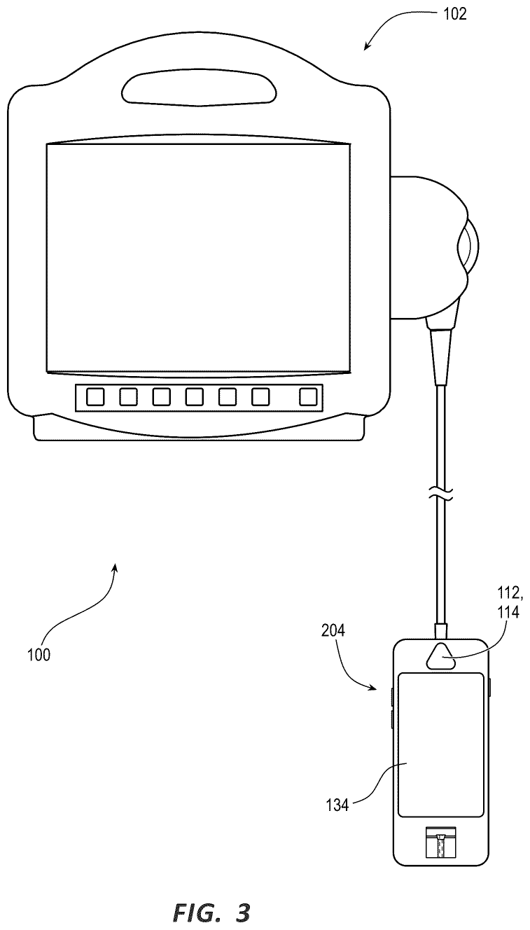

FIGS. 1 and 3 illustrate an ultrasound system 100 including a console 102 and either a first ultrasound probe 104 or a second ultrasound probe 204 in accordance with some embodiments.

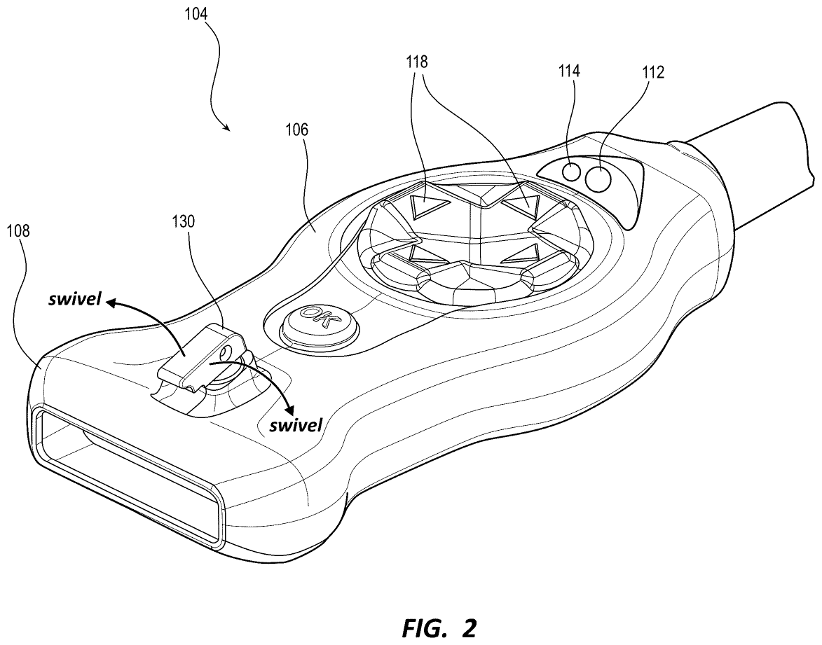

FIG. 2 illustrates a perspective view of the ultrasound probe 104 in accordance with some embodiments.

As shown, the ultrasound probe 104 includes a probe body 106, a probe head 108 extending from a distal end of the probe body 106, and a plurality of ultrasonic transducers 110 arranged in an array in the probe head 108.

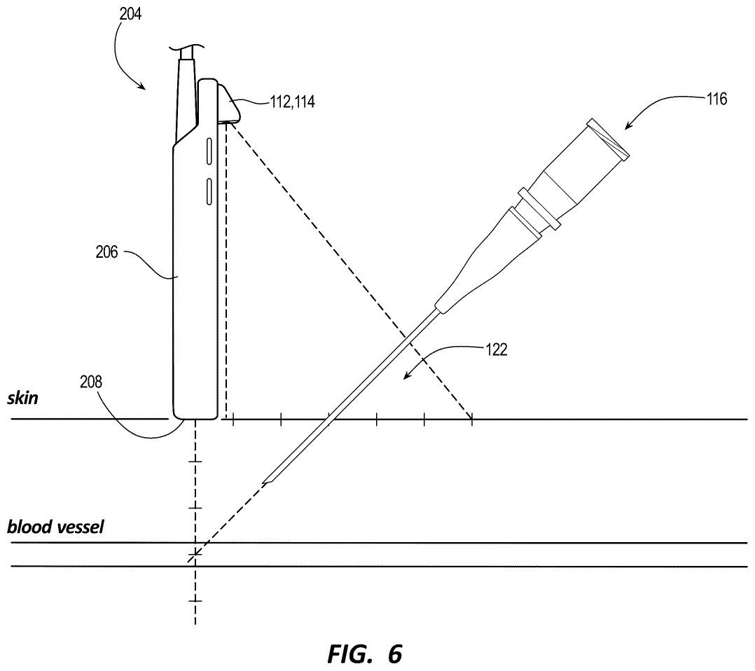

The ultrasound probe 104 can also include a camera 112 integrated into a side of the ultrasound probe 104, a light-pattern projector 114 (e.g., a laser light-pattern projector) integrated into the side of the ultrasound probe 104, or both the camera 112 and the light-pattern projector 114 integrated into the side of the ultrasound probe 104. Notably, the side of the ultrasound probe 104 including the camera 112 or the light-pattern projector 114 is shown in FIG. 2 as a major side of the ultrasound probe 104, specifically a top side (or front face) of the ultrasound probe 104, which is convenient for an out-of-plane view of a needle 116 (see FIG. 6) when establishing an insertion site with the needle 116 as set forth in the method below. In addition, the foregoing side of the ultrasound probe 104 conveniently includes various buttons 118 of the ultrasound probe 104 useful for operating the ultrasound probe 104 or 204 or the ultrasound system 100 while establishing an insertion site with the needle 116. That said, the side of the ultrasound probe 104 including the camera 112 or the light-pattern projector 114 can alternatively be a minor side of the ultrasound probe 104, which is convenient for an in-plane view of the needle 116 when establishing an insertion site with the needle 116 as set forth in the method below.

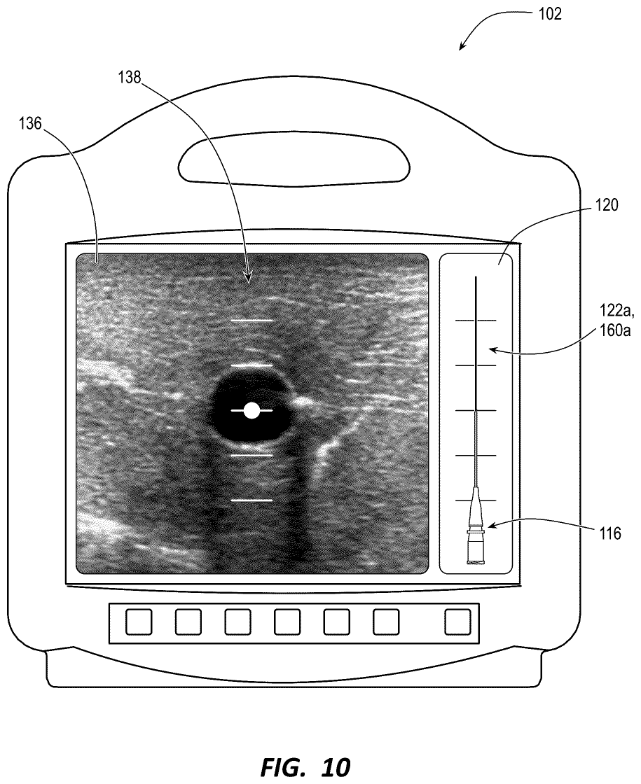

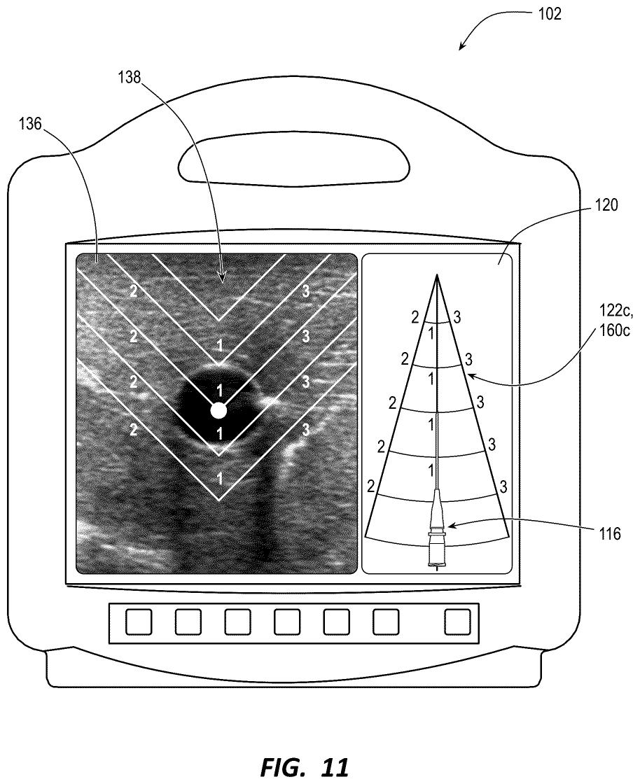

The camera 112 is configured for recording one or more still or moving images 120 (see FIGS. 10 and 11) of a procedural field including a subject portion of a patient therein with a depth of field including a plane of a distal end of the probe head 108 and a field of view including a spatial region about the probe head 108. As set forth in more detail below, the one-or-more still or moving images 120 can be rendered on the display screen of the display 158 along with the ultrasound images 136 associated therewith, which allows a clinician to sustain spatial attention on the display 158 when establishing an insertion site with the needle 116, thereby obviating the clinician from frequently switching his or her spatial attention between the display 158 and the procedural field as done with existing ultrasound systems.

The light-pattern projector 114 is configured to project a light pattern 122 in the spatial region about the probe head 108 focused in the plane of the distal end of the probe head 108, thereby including the foregoing subject portion of the patient in the procedural field. The light pattern 122 is configured for guided insertion of the needle 116 into an anatomical target under the probe head 108 in the procedural field. Similar to the one-or-more still or moving images 120 when rendered on the display screen of the display 158, the light pattern 122 when projected in the spatial region about the probe head 108 allows a clinician to sustain spatial attention in the procedural field when establishing an insertion site with the needle 116 as set forth in the method below, thereby obviating the clinician from frequently switching his or her spatial attention between the procedural field and the display 158 as done with existing ultrasound systems.

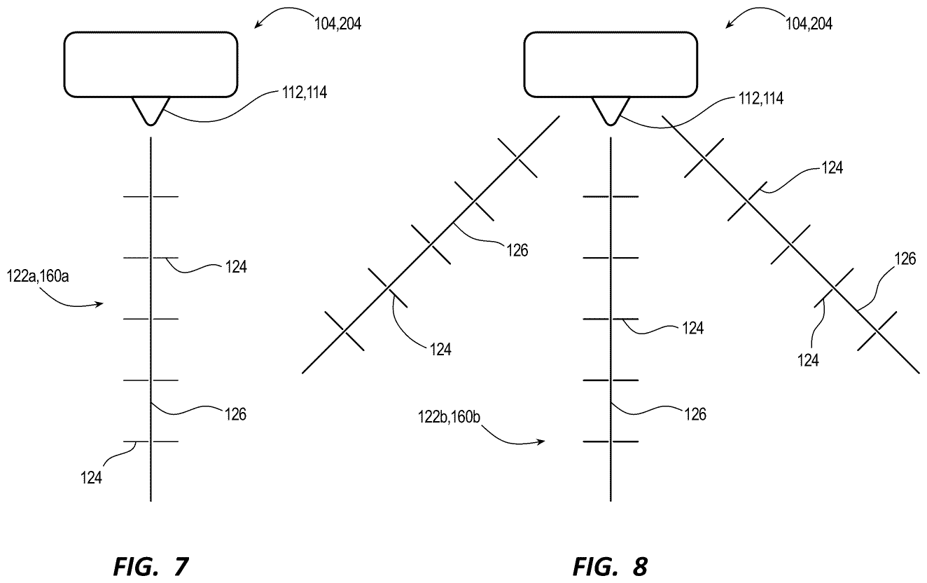

FIG. 7 illustrates a schematic of a first light pattern 122a in accordance with some embodiments. FIG. 8 illustrates a schematic of a second light pattern 122b in accordance with some embodiments. Notably, when referring to a generic light pattern herein, the light pattern 122 is referenced. When referring to a specific light pattern herein, the light pattern 122a, 122b, or the like is referenced.

As shown, the light pattern 122a of 122b includes periodic hash marks 124 along one or more rays 126 radiating from a central axis of the ultrasound probe 104 in the plane of the probe head 108. Indeed, the light pattern 122a includes the hash marks 124 along one ray 126 radiating from the central axis of the ultrasound probe 104, whereas the light pattern 122b includes the hash marks 124 along three rays 126 radiating from the central axis of the ultrasound probe 104. As shown in FIG. 6, each hash mark of the hash marks 124 corresponds to a depth under the probe head 108 accessible by the needle 116 along an associated ray 126 at a needle-insertion angle with respect to the plane of the probe head 108.

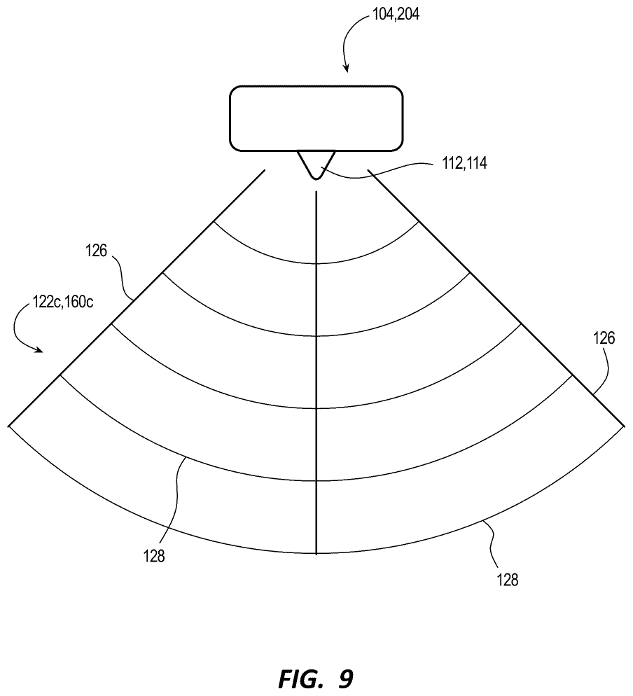

FIG. 9 illustrates a schematic of a third light pattern 122c in accordance with some embodiments.

As shown, the light pattern 122c includes periodic concentric circular arcs 128 bound between two or more rays 126 radiating from the central axis of the ultrasound probe 104 in the plane of the probe head 108. Indeed, the light pattern 122c includes the circular arcs 128 bound between three rays 126 radiating from the central axis of the ultrasound probe 104. As shown in FIG. 6, each circular arc of the circular arcs 128 corresponds to a depth under the probe head 108 accessible by the needle 116 along an associated ray 126 at a needle-insertion angle with respect to the plane of the probe head 108. Notably, the associated ray 126 can be an intervening ray between the two-or-more rays 126 of the light pattern 122c radiating from the central axis of the ultrasound probe 104. The intervening ray need not be a visible ray of the light pattern 122c; the intervening ray can be envisioned between the two-or-more rays 126 of the light pattern 122c and followed with the needle 116 when establishing an insertion site therewith as set forth in the method below.

The ultrasound probe 104 can also include a needle-guide holder 130 extending from the side of the probe head 108 in common with the side of the ultrasound probe 104 including the camera 112, whether the foregoing side is the major or minor side of the ultrasound probe 104 including the camera 112 or the light-pattern projector 114.

The ultrasound probe 104 can also include a single-use needle guide 132 configured to couple to the needle-guide holder 130. The needle guide 132, the needle-guide holder 130, or a combination of the needle guide 132 and the needle-guide holder 130 can include at least one degree of freedom enabling the needle guide 132 to swivel between sides of the ultrasound probe 104. Indeed, the needle guide 132 can swivel between minor sides of the ultrasound probe 104 if the needle-guide holder 130 extends from a major side of the ultrasound probe 104. The needle guide 132 can alternatively swivel between major sides of the ultrasound probe 104 if the needle-guide holder 130 extends from a minor side of the ultrasound probe 104. To enable the needle guide 132 to swivel between the foregoing sides of the ultrasound probe 104, the needle guide 132 and the needle-guide holder 130 can include a joint (e.g., ball joint) formed therebetween that provides the degree of freedom needed. If the needle guide 132 is used with the needle 116 to establish an insertion site, the needle guide 132 can be advantageously swiveled along each circular arc of the circular arcs 128 of the light pattern 122c. The needle 116 can be subsequently inserted along any existing or envisioned ray of the light pattern 122c to establish an insertion site.

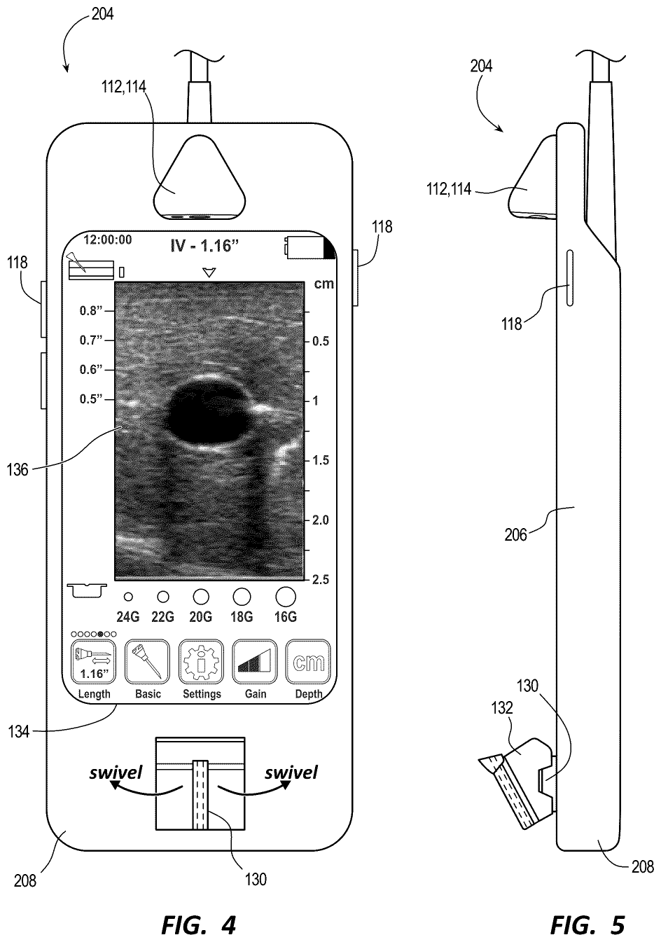

FIGS. 4 and 5 illustrate different views of the ultrasound probe 204 in accordance with some embodiments.

As shown, the ultrasound probe 204 includes a probe body 206, a probe head 208 extending from a distal end of the probe body 206, and the plurality of ultrasonic transducers 110 arranged in an array in the probe head 208. In addition, the ultrasound probe 204 can include the camera 112 integrated into a side of the ultrasound probe 204, the light-pattern projector 114 integrated into the side of the ultrasound probe 204, or both the camera 112 and the light-pattern projector 114 integrated into the side of the ultrasound probe 204. As such, the ultrasound probe 204 is like the ultrasound probe 104 in certain ways. Therefore, the description set forth above for the ultrasound probe 104 likewise applies to the ultrasound probe 204.

The ultrasound probe 204 also includes a display 134 integrated into the side of the ultrasound probe 204, specifically the top side (or front face) of the ultrasound probe 204, which differentiates the ultrasound probe 204 from the ultrasound probe 104. The display 134 is configured to render ultrasound images 136 on a display screen thereof, which allows a clinician to sustain spatial attention in the procedural field when establishing an insertion site with the needle 116 as set forth in the method below, thereby obviating the clinician from frequently switching his or her spatial attention between the procedural field, which includes the display 134, and another display (e.g., the display 158 of the console 102) as done with existing ultrasound systems. In addition, the display 134 is configured to render one or more overlying needle trajectories 138 over the ultrasound images 136. (See, for example, FIG. 11 for the one-or-more needle trajectories 138.) The one-or-more needle trajectories 138 are configured for guided insertion of the needle 116 into an anatomical target under the probe head 208 on the display 134. Indeed, the one-or-more needle trajectories 138 are in accordance with one or more depths accessible by the needle 116 as indicated by the light pattern 122.

Notably, the ultrasound probe 104 or 204 can include magnetic sensors to enhance guided insertion of the needle 116 into an anatomical target as set forth herein with magnetic-based needle guidance. Such magnetic-based needle guidance is disclosed in U.S. Pat. Nos. 8,388,541; 8,781,555; 8,849,382; 9,456,766; 9,492,097; 9,521,961; 9,554,716; 9,636,031; 9,649,048; 10,449,330; 10,524,691; and 10,751,509, each of which is incorporated by reference in its entirety into this application.

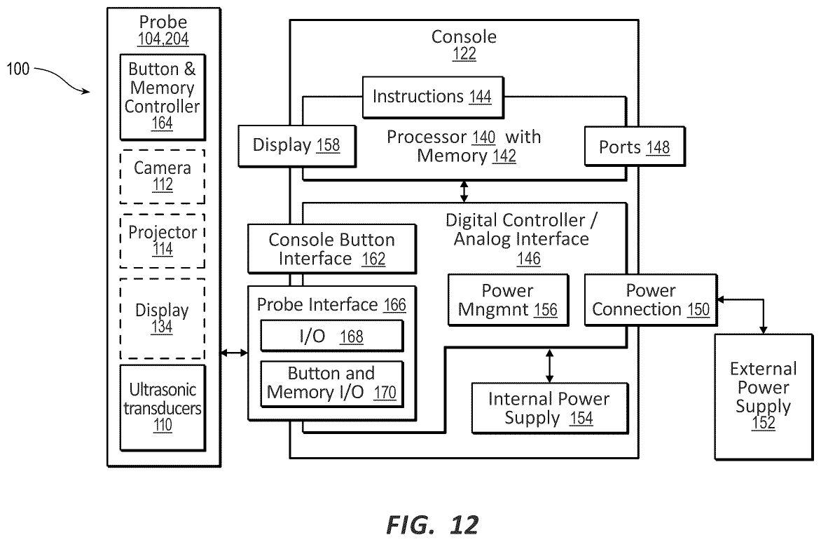

FIG. 12 illustrates a block diagram of the ultrasound system 100 in accordance with some embodiments.

As shown, the console 102 includes a variety of components including a processor 140 and memory 142 such as random-access memory (“RAM”) or non-volatile memory (e.g., electrically erasable programmable read-only memory [“EEPROM”]) for controlling various functions of the ultrasound system 100 during operation thereof. Indeed, the console 102 is configured to instantiate by way of executable instructions 144 stored in the memory 142 and executed by the processor 140 various processes for controlling the various functions of the ultrasound system 100.

As to the various processes for controlling the various functions of the ultrasound system 100, the various processes can include beamforming by way of a beamformer configured to drive the ultrasonic transducers 110, wherein driving the ultrasonic transducers 110 includes emitting generated ultrasound signals as well as receiving, amplifying, and digitizing reflected ultrasound signals; signal processing by way of a signal processor configured to detect an amplitude of each of the foregoing reflected ultrasound signals or the digitized signals corresponding thereto; and image processing by way of an image processor configured to manage storage of detected amplitudes and send the ultrasound images 136 corresponding to collections of the detected amplitudes to the display screen of the display 134 or 158 upon completion of the ultrasound images 136.

Further to the various processes for controlling the various functions of the ultrasound system 100, the various processes can include processing electrical signals corresponding to color and brightness data from an image sensor of the camera 112 of the ultrasound probe 104 or 204 into the one-or-more still or moving images 120; determining depths for various anatomical structures in the ultrasound images 136 by way of delays in time between emitting the generated ultrasound signals from the ultrasonic transducers 110 and receiving the reflected ultrasound signals by the ultrasonic transducers 110; adjusting a scale of the light pattern 122 projected from the light-pattern projector 114 in accordance with both the depths for the various anatomical structures in the ultrasound images 136 and a needle-insertion angle, wherein the needle-insertion angle is selected from a single ultrasound system-defined needle-insertion angle, a clinician-selected needle-insertion angle among various ultrasound system-defined needle-insertion angles, and a dynamic needle-insertion angle determined by way of magnetic-based needle guidance; adjusting a scale of the overlying pattern 160 lying over the one-or-more still or moving images 120 in accordance with both the depths for the various anatomical structures in the ultrasound images 136 and the needle-insertion angle; and adjusting a scale of the one-or-more needle trajectories 138 lying over the ultrasound images 136 in accordance with both the depths for various anatomical structures in the ultrasound images 136 and the needle-insertion angle.

The console 102 also includes a digital controller/analog interface 146 in communication with both the processor 140 and other system components to govern interfacing between the ultrasound probe 104 or 204 and the foregoing system components. Ports 148 are also included in the console 102 for connection with additional system components including optional system components such as a printer, storage media, a keyboard, etc. The ports 148 can be universal serial bus (“USB”) ports, though other types of ports can be used for these connections or any other connections shown or described herein.

A power connection 150 is included with the console 102 to enable an operable connection to an external power supply 152. An internal power supply 154 (e.g., a battery) can also be employed either with or exclusive of the external power supply 152. Power management circuitry 156 is included with the digital controller/analog interface 146 of the console 102 to regulate power use and distribution.

A display 158 integrated into the console 102 is configured to render on a display screen thereof a graphical user interface (“GUI”), the ultrasound images 136 attained by the ultrasound probe 104 or 204, the one-or-more still or moving images 120 of the procedural field attained by the camera 112 of the ultrasound probe 104 or 204, an overlying pattern 160 lying over the one-or-more still or moving images 120, the one-or-more needle trajectories 138 lying over the ultrasound images 136, etc. That said, the display 158 can alternatively be separate from the console 102 and communicatively coupled thereto. Regardless, control buttons (see FIGS. 1, 3, 10, and 11) accessed through a console button interface 162 of the console 102 can be used to immediately call up to the display screen a desired mode of the ultrasound system 100 for assisting with an ultrasound-based medical procedure such as that for establishing an insertion site with the needle 116, placing a VAD such as a catheter in a blood vessel of a patient at the insertion site, or the like. For example, a mode of the ultrasound system 100 for establishing an insertion site with the needle 116 can include rendering the one-or-more still or moving images 120 of the procedural field, the overlying pattern 160 lying over the one-or-more still or moving images 120, the one-or-more needle trajectories 138 lying over the ultrasound images 136, or a combination thereof.

FIGS. 10 and 11 illustrate guided insertion of the needle 116 into an anatomical target of an ultrasound image with the light pattern 122, specifically the light pattern 122a and 122c, respectively, as shown in the one-or-more still or moving images 120 adjacent the ultrasound image on the display 158.

When rendered on the display screen, the one-or-more still or moving images 120 show at least the needle 116 when the needle 116 is present in the spatial region about the probe head 108 or 208, which, even alone, allows a clinician to sustain spatial attention on the display 158 when establishing an insertion site with the needle 116. If the ultrasound probe 104 or 204 includes the light-pattern projector 114, however, the one-or-more still or moving images 120 can show both the light pattern 122 in the spatial region about the probe head 108 or 208 and the needle 116 in relation to the light pattern 122 for guided insertion of the needle 116 into an anatomical target under the probe head 108 or 208 on the display 158. Having both the light pattern 122 and the needle 116 shown in the one-or-more still or moving images 120 further allows a clinician to sustain spatial attention on the display 158 when establishing the insertion site with the needle 116, thereby obviating the clinician from frequently switching his or her spatial attention between the display 158 and the procedural field as done with existing ultrasound systems.

FIGS. 10 and 11 also illustrate guided insertion of the needle 116 into an anatomical target of an ultrasound image respectively with the overlying pattern 160, specifically the overlying pattern 160a and 160c, respectively, over the one-or-more still or moving images 120 adjacent the ultrasound image on the display 158.

Following on the foregoing, if the ultrasound probe 104 or 204 does not include the light-pattern projector 114, or if a clinician prefers not to use the light-pattern projector 114 of the ultrasound probe 104 or 204, the one-or-more still or moving images 120 can show the overlying pattern 160 lying thereover. When the needle 116 is present in the spatial region about the probe head 108 or 208, the one-or-more still or moving images 120 can thusly show both the overlying pattern 160 and the needle 116 in relation to the overlying pattern 160 for guided insertion of the needle 116 into an anatomical target under the probe head 108 or 208 on the display 158. Having both the overlying pattern 160 and the needle 116 shown in the one-or-more still or moving images 120 further allows a clinician to sustain spatial attention on the display 158 when establishing the insertion site with the needle 116, thereby obviating the clinician from frequently switching his or her spatial attention between the display 158 and the procedural field as done with existing ultrasound systems.

Like the light pattern 122a or 122b, the overlying pattern 160a or 160b includes the periodic hash marks 124 along one or more rays 126 radiating from the central axis of the ultrasound probe 104 or 204 in the plane of the probe head 108 or 208; however, unlike the light pattern 122a or 122b, the hash marks 124 and the one-or-more rays 126 are virtual, existing only on the display screen. By analogy to the light pattern 122a, the overlying pattern 160a likewise includes the hash marks 124 along one ray 126 radiating from the central axis of the ultrasound probe 104 or 204, and, by analogy to the light pattern 122b, the overlying pattern 160b likewise includes the hash marks 124 along three rays 126 radiating from the central axis of the ultrasound probe 104 or 204. Each hash mark of the hash marks 124 corresponds to a depth under the probe head 108 or 208 accessible by the needle 116 along an associated ray 126 at a needle-insertion angle with respect to the plane of the probe head 108 or 208.

Like the light pattern 122c, the overlying pattern 160c includes periodic concentric circular arcs 128 bound between two or more rays 126 radiating from a central axis of the ultrasound probe 104 or 204 in the plane of the probe head 108 or 208; however, unlike the light pattern 122c, the circular arcs 128 and the two-or-more rays 126 are virtual, existing only on the display screen. By analogy to the light pattern 122c, the overlying pattern 160c likewise includes the circular arcs 128 bound between three rays 126 radiating from the central axis of the ultrasound probe 104 or 204. Each circular arc of the circular arcs 128 corresponds to a depth under the probe head 108 or 208 accessible by the needle 116 along an associated ray 126 at a needle-insertion angle with respect to the plane of the probe head 108 or 208. Notably, the associated ray 126 can be an intervening ray between the two-or-more rays 126 of the overlying pattern 160c radiating from the central axis of the ultrasound probe 104 or 204. The intervening ray need not be a visible ray of the overlying pattern 160c; the intervening ray can be envisioned between the two-or-more rays 126 of the overlying pattern 160c and followed with the needle 116 when establishing an insertion site therewith as set forth in the method below.

As set forth above, the display 158 is configured to render on the display screen thereof the one-or-more needle trajectories 138 lying over the ultrasound images 136. The one-or-more needle trajectories 138 are configured for guided insertion of the needle 116 into an anatomical target under the probe head 108 or 208 on the display 158. Indeed, as shown in FIG. 11, the one-or-more needle trajectories 138 are in accordance with one or more depths accessible by the needle 116 indicated by the light pattern 122c or the overlying pattern 160c.

The needle trajectories 138 labeled ‘1’ in FIG. 11 are straightforwardly understood as being in a plane perpendicular to that of an ultrasound beam for a so called out-of-plane view with respect to the needle 116. Moving the needle 116 from circular arc 128 to circular arc 128 of the light pattern 122c or overlying pattern 160c of FIG. 11 toward the central axis of the ultrasound probe 104 or 204 while keeping the needle-insertion angle constant moves the needle 116 from trajectory to trajectory of the one-or-more needle trajectories 138 in a same direction (e.g., up) on the display screen. Indeed, inserting the needle 116 into a patient at the circular arc 128 nearest the central axis of the ultrasound probe 104 or 204 results in overshooting an anatomical target, for example, a blood vessel under the probe head 108 or 208. Notably, the needle 116 could still access the blood vessel but distal of the probe head 108 or 208. Similarly, moving the needle 116 from circular arc 128 to circular arc 128 of the light pattern 122c or overlying pattern 160c of FIG. 11 away from the central axis of the ultrasound probe 104 or 204 while keeping the needle-insertion angle constant moves the needle 116 from trajectory to trajectory of the one-or-more needle trajectories 138 in a same direction (e.g., down) on the display screen. Indeed, inserting the needle 116 into the patient at the circular arc 128 farthest from the central axis of the ultrasound probe 104 or 204 results in undershooting the blood vessel under the probe head 108 or 208. Notably, the needle 116 would still access the blood vessel but proximal of the probe head 108 or 208 and, ultimately, through a backwall of the blood vessel if the needle trajectory is completely followed.

The needle trajectories 138 labeled ‘2’ and ‘3’ in of FIG. 11 are in mirrored planes oblique to that of the ultrasound beam, and, as such, approach the blood vessel obliquely. However, like that set forth for the needle trajectories 138 labeled ‘1’ in FIG. 11, moving the needle 116 from circular arc 128 to circular arc 128 of the light pattern 122c or overlying pattern 160c of FIG. 11 toward the central axis of the ultrasound probe 104 or 204 while keeping the needle-insertion angle constant moves the needle 116 from trajectory to trajectory of the one-or-more needle trajectories 138 in a same direction (e.g., up) on the display screen. Moving the needle 116 from circular arc 128 to circular arc 128 of the light pattern 122c or overlying pattern 160c of FIG. 11 away from the central axis of the ultrasound probe 104 or 204 while keeping the needle-insertion angle constant moves the needle 116 from trajectory to trajectory of the one-or-more needle trajectories 138 in a same direction (e.g., down) on the display screen.

Adverting briefly back to the ultrasound probe 104 or 204, the ultrasound probe 104 or 204 includes the buttons 118 for operating the ultrasound probe 104 or 204 or the ultrasound system 100 of which the ultrasound probe 104 or 204 is part. For example, the buttons 118 can be configured for selecting a desired mode of the ultrasound system 100 as set forth above. The ultrasound probe 104 or 204 includes a button-and-memory controller 164 configured for operable communication with a probe interface 166 of the console 102, which probe interface 166 includes an input/output (“I/O”) component 168 for interfacing with the ultrasonic transducers 110 and a button-and-memory I/O component 170 for interfacing with the button-and-memory controller 164.

### Methods

Methods include a method of using the ultrasound system 100 to establish an insertion site for access to an anatomical structure (e.g., blood vessel) of a patient. The method includes one or more steps selected from an ultrasound probe-obtaining step, an ultrasound probe-moving step, a recording step, an ultrasound image-monitoring step, a needle guide-attaching step, a needle guide-swiveling step, and a needle-inserting step.

The ultrasound probe-obtaining step includes obtaining the ultrasound probe 104. As set forth above, the ultrasound probe 104 includes the probe body 106, the probe head 108 extending from the distal end of the probe body 106, and the camera 112 integrated into the side of the ultrasound probe 104.

The needle guide-attaching step includes attaching the needle guide 132 to the needle-guide holder 130 extending from the probe body 106. The needle guide 132 includes a needle through hole configured to direct the needle 116 into the patient under the probe head 108 at the needle-insertion angle defined by the needle guide 132.

The ultrasound probe-moving step includes moving the ultrasound probe 104 over the patient while the ultrasound probe 104 emits generated ultrasound signals into the patient from the ultrasonic transducers 110 in the probe head 108 and receives reflected ultrasound signals from the patient by the ultrasonic transducers 110.

The recording step includes recording the one-or-more still or moving images 120 of the procedural field including a subject portion of the patient therein. As set forth above, the one-or-more still or moving images 120 are recorded with a depth of field including the plane of the distal end of the probe head 108 and the field of view including the spatial region about the probe head 108.

The ultrasound image-monitoring step includes monitoring ultrasound images 136 rendered on the display screen of the display 158 associated with the console 102 of the ultrasound system 100 to identify an anatomical target of the patient under the probe head 108.

The needle guide-swiveling step includes swiveling the needle guide 132 between sides of the ultrasound probe 104 to find a suitable needle trajectory before the needle-inserting step. The needle-guide holder 130, the needle guide 132, or a combination of the needle-guide holder 130 and the needle guide 132 such as the joint formed therebetween includes at least one degree of freedom enabling the swiveling of the needle guide 132.

The needle-inserting step includes inserting the needle 116 into the anatomical target. The inserting of the needle 116 into the anatomical target during the needle-inserting step is guided in the procedural field with reference to the light pattern 122 in the spatial region about the probe head 108, on the display 158 with reference to the one-or-more still or moving images 120 or the one-or-more needle trajectories 138 rendered on the display screen thereof, or a combination thereof.

As to guidance in the procedural field with reference to the light pattern 122, the light pattern 122 is projected into the spatial region about the probe head 108 from the light-pattern projector 114 and focused in the plane of the distal end of the probe head 108 for guiding the needle 116 in the procedural field. As set forth above, the light pattern 122a or 122b includes the periodic hash marks 124 along the one-or-more rays 126 radiating from the central axis of the ultrasound probe 104 in the plane of the probe head 108. Each hash mark of the hash marks 124 corresponds to a depth under the probe head 108 accessible by the needle 116 along an associated ray 126 at a needle-insertion angle with respect to the plane of the probe head 108. As further set forth above, the light pattern 122c includes the periodic concentric circular arcs 128 bound between the two-or-more rays 126 radiating from the central axis of the ultrasound probe 104 in the plane of the probe head 108. Each circular arc of the circular arcs 128 corresponds to a depth under the probe head 108 accessible by the needle 116 along an associated ray 126 at a needle-insertion angle with respect to the plane of the probe head 108.

As to guidance on the display 158 with reference to the one-or-more still or moving images 120, the one-or-more still or moving images 120 can show both the light pattern 122 in the spatial region about the probe head 108 and the needle 116 in relation to the light pattern 122 for guiding the needle 116 on the display 158. However, if the ultrasound probe 104 does not include the light-pattern projector 114, or if a clinician prefers not to use the light-pattern projector 114 of the ultrasound probe 104, the one-or-more still or moving images 120 can show the overlying pattern 160 lying thereover for guiding the needle 116 on the display 158. As set forth above, the overlying pattern 160a or 160b includes the periodic hash marks 124 along the one-or-more rays 126 radiating from the central axis of the ultrasound probe 1014 in the plane of the probe head 108. Each hash mark of the hash marks 124 corresponds to a depth under the probe head 108 accessible by the needle 116 along an associated ray 126 at a needle-insertion angle with respect to the plane of the probe head 108. As further set forth above, the overlying pattern 160c includes the periodic concentric circular arcs 128 bound between the two-or-more rays 126 radiating from the central axis of the ultrasound probe 104 in the plane of the probe head 108. Each circular arc of the circular arcs 128 corresponds to a depth under the probe head 108 accessible by the needle 116 along an associated ray 126 at a needle-insertion angle with respect to the plane of the probe head 108.

Further as to guidance on the display 158 with reference to the one-or-more needle trajectories 138, the ultrasound images 136 can show the one-or-more needle trajectories 138 in accordance with one or more depths accessible by the needle 116 indicated by the light pattern 122 or overlying pattern 160 in the one-or-more still or moving images 120 for guiding the needle 116 on the display 158.

Notably, the foregoing method involves the ultrasound probe 104; however, the method can be modified for the ultrasound probe 204. In such a method, the ultrasound images 136 are displayed on the display 134 of the ultrasound probe 204, optionally, in combination with the ultrasound images 136 and the one-or-more still or moving images 120 on the display 158 of the console 102. As set forth above, displaying the images on the display 134 of the ultrasound probe 204 allows a clinician to sustain spatial attention in the procedural field when establishing the insertion site with the needle 116 in the needle-inserting step, thereby obviating the clinician from frequently switching his or her spatial attention between the procedural field, which includes the display 134, and another display (e.g., the display 158 of the console 102) as done with existing ultrasound systems.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. An ultrasound probe, comprising:
a display screen on a visible side of the ultrasound probe, the display screen coupled to ultrasound imaging components in the ultrasound probe, the ultrasound imaging components configured to capture live subcutaneous images and render the live subcutaneous images on the display screen;
one or more needle trajectories depicted on the display over the live subcutaneous images, the one or more needle trajectories configured to assist a user in guided insertion of a needle into an anatomical target under the ultrasound probe; and
a light-pattern projector on the visible side of the ultrasound probe adjacent the display screen, the light-pattern projector configured to project a light pattern corresponding to a subcutaneous depth accessible by a needle.

2. The ultrasound probe according to claim 1, wherein the light pattern comprises a plurality of hash marks along a beam of light, each of the plurality of hash marks corresponding to the subcutaneous depth accessible by the needle.

3. The ultrasound probe according to claim 1, wherein the light pattern comprises concentric circular arcs bound between beams of light, each of the concentric circular arcs corresponding to the subcutaneous depth accessible by the needle.

4. The ultrasound probe according to claim 3, wherein the one or more needle trajectories are in accordance with concentric circular arcs.

5. The ultrasound probe according to claim 1, further comprising a needle-guide holder on the visible side of the ultrasound probe adjacent the display screen.

6. The ultrasound probe according to claim 5, further comprising a single-use needle guide coupled to the needle-guide holder, the needle guide including at least one degree of freedom enabling the needle guide to swivel.

7. The ultrasound probe according to claim 1, further comprising a camera configured for recording images of a procedural field with a depth of field including a plane of a skin contacting end of the ultrasound probe and a field of view including a spatial region around the skin contacting end of the ultrasound probe.

8. The ultrasound probe according to claim 7, wherein the camera is positioned adjacent the light-pattern projector on the visible side of the ultrasound probe.

9. The ultrasound probe according to claim 7, wherein the images show both the light pattern in the spatial region around the skin contacting end of the ultrasound probe and the needle in relation to the light pattern when both the light pattern and the needle are present in the spatial region around the skin contacting end of the ultrasound probe.

10. The ultrasound probe according to claim 1, further comprising depth markings on each side of the display screen, wherein a first set of depth markings on a first side are marked in inches, and wherein a second set of depth markings on a second side opposite the first side are marked in centimeters.

11. The ultrasound probe according to claim 10, further comprising a selection of needle gauge sizes adjacent the display screen.

12. The ultrasound probe according to claim 11, further comprising a selection of needle lengths adjacent the display screen.

13. The ultrasound probe according to claim 12, further comprising a selection of needle types adjacent the display screen.

14. The ultrasound probe according to claim 13, wherein a selected needle gauge, a selected needle length, and a selected needle type are indicated adjacent the display screen.
